(19) United States
(12) Patent Application Publication
(10) Pub. No.: US 2008/0227098 A1
(43) Pub. Date: Sep. 18, 2008

(54) METHOD FOR DIAGNOSING CANCER OF THE PROSTATE

(75) Inventors: Maryla Krajewska, La Jolla, CA (US); John C. Reed, La Jolla, CA (US); Stan Krajewski, La Jolla, CA (US)

Correspondence Address: KNOBBE MARTENS OLSON & BEAR LLP, 2040 MAIN STREET, FOURTEENTH FLOOR, IRVINE, CA, 92614, US

(73) Assignee: Bumham Institute for Medical Research, La Jolla, CA (US)

(21) Appl. No.: 12/031,639

(22) Filed: Feb. 14, 2008

Related U.S. Application Data
(60) Provisional application No. 60/889,877, filed on Feb. 14, 2007.

(51) Int. Cl. C12Q 1/68; G01N 33/00; G01N 33/566; G01N 33/574; C12Q 1/02

(52) U.S. Cl. 435/6; 436/86; 436/501; 435/29; 435/7.23

(57) ABSTRACT

Methods for diagnosis and prognosis of prostate cancer are provided. The methods involve the detection of the level of expression of Claudin-1 in tissue or cell samples. Claudin-1 is underexpressed or non-expressed in the majority of prostate cancers.

### CROSS-REFERENCE TO RELATED APPLICATIONS

This application claims the benefit of priority under 35 U.S.C. §119 to U.S. Provisional Patent Application No. 60/889,877 filed Feb. 14, 2007, and which is expressly incorporated herein by reference in its entirety.

### STATEMENT REGARDING FEDERALLY SPONSORED R&D

This work was partly covered by NIH SPEC Consortium grant: CA 114810-02. The US Government has certain rights in this invention.

### BACKGROUND OF THE INVENTION

1. Field of the Invention

The invention relates to the fields of molecular biology and medicine. In particular, disclosed herein are methods for detecting prostate cancer in a patient using Claudin-1 protein alone or in combination with alpha-methylacyl-CoA racemas (AMACR).

2. Description of the Related Art

With the increasing life expectancy, carcinoma of the prostate represents a high proportion of all new cancers in men in the United States. Of the more than 700,000 new cancer cases registered in men each year, approximately one third were projected to be cancer of the prostate in 2005 (reviewed in Chodak G. 2006 Rev. Urol. 8:S3-8). Prostate-Specific Antigen (PSA) testing facilitates early diagnosis of prostate cancer, but up to 60% of PSA tests are false-positive, making histological confirmation of diagnosis critical. Skinny needle biopsy is prone to sampling errors and inconsistent interpretations, suggesting a need for complementary diagnostic methods that provide more accurate diagnosis.

Claudins are a family of more than 20 integral membrane tight junction (TJ) proteins that maintain cell polarity and paracellular barrier functions in epithelial and endothelial cells (reviewed in Swisshelm K. et al. 2005 Adv. Drug Deliv. Rev. 57:919-928).

Claudin-1 was identified as a novel mouse integral membrane protein localizing at tight junctions (Furuse M. et al. 1998 Cell Biol. 141:1539-1550), and its expression is reduced in proliferating compared to quiescent epithelial cells (Swisshelm K. et al. 1999 Gene 226:285-295). Moreover, reduced Claudin-1 expression has been reported for various cancers (reviewed in Morin P. J. 2005 Cancer Res. 65:9603-9606), though not previously explored in prostate cancer.

### SUMMARY OF THE INVENTION

Embodiments disclosed herein relate to methods for diagnosing prostate cancer in a patient. Some approaches include providing a patient at risk for prostate cancer, determining a level of Claudin-1 expression in a prostate sample from the patient, and assessing whether Claudin-1 is expressed at a level which is lower than a predetermined level in normal, non-cancerous prostate (e.g., normal epithelial glands, glandular hyperplasia; e.g. BPH, cystically-dilated glands) or precancerous changes (dysplasia), thereby diagnosing prostate cancer in the patient. In some embodiments, a prostate sample, e.g., a biopsy tissue sample, can be obtained from the patient. Other embodiments include grading the prostate sample such that the grade is high if Claudin-1 expression is low.

Further embodiments disclosed herein relate to methods for diagnosing prostate cancer in a patient including providing a patient at risk for prostate cancer, determining a level of Claudin-1 expression in a prostate sample from the patient, determining a level of expression of alpha-methylacyl-CoA racemas (AMACR) in the prostate sample from the patient, and assessing whether Claudin-1 is expressed at a level which is lower tan a predetermined level in normal, non-cancerous prostate (e.g., normal epithelial glands, glandular hyperplasia; e.g. BPH, cystically-dilated glands) or precancerous changes (dysplasia) and whether AMACR is expressed at a level that is higher than a predetermined level, thereby diagnosing prostate cancer (e.g., florid prostate cancer) or prostatic intraepithelial neoplasia (PIN) in the patient.

Additional embodiments disclosed herein relate to methods for monitoring the progress of a prostate cancer therapy in a subject. Some approaches include identifying a subject with prostate cancer, providing the subject a prostate cancer therapy, administering to the subject a detectable amount of a Claudin-1 antibody that comprises a detectable label, and determining the presence or amount of Claudin-1 antibody bound to prostate cancer cells in the subject, before a treatment with the prostate cancer therapy and during or after a period of the treatment.

More embodiments disclosed herein relate to kits that includes an agent that specifically detects Claudin-1 expression. The kit may further include instruction for using the kit components. The kit may also include an agent that specifically detects AMACR expression.

In some embodiments, the level of Claudin-1 and/or AMACR expression can be determined by contacting the prostate sample with an anti-Claudin-1 antibody and/or an anti-AMACR antibody, respectively. Anti-Claudin-1 antibodies and/or anti-AMACR antibodies can be polyclonal or monoclonal antibodies. In other embodiments, the level of Claudin-1 and/or AMACR expression can be determined by determining the level of Claudin-1 and/or AMACR mRNA.

### BRIEF DESCRIPTION OF THE DRAWINGS

FIG. 1 is a panel of photographs of normal prostate specimens contained on tissue microarrays contacted by antibodies against Claudin-1, HMW cytokeratin, and/or p63 and detected by bright-field and chromagen based indirect immunocytochemistry (ICH).

FIG. 2 is a panel of photographs of normal and malignant prostate specimens contained on tissue microarrays contacted by antibodies against Claudin-1, HMW cytokeratin, and/or p63 and detected by bright-field and chromagen based indirect immunocytochemistry (ICH).

### DETAILED DESCRIPTION OF THE PREFERRED EMBODIMENT

Embodiments disclosed herein diagnostic and prognostic methods for the detection of prostate cancer. The methods relate to the discovery that Claudin-1 is differentially expressed in prostate cancer cells. In particular, Claudin-1 is underexpressed in prostate cancer cells and tissue, compared to normal prostate cells and tissue. Thus, the detection of the expression (or lack thereof) of Claudin-1 provides a means of determining whether or not cells or tissue from the prostate are malignant. Such detection methods may be used, for example, for early diagnosis of the disease, to monitor the progress of the disease or the progress of treatment protocols, or to assess the grade of the cancer.

The terms “individual,” “host,” “subject,” and “patient” are used interchangeably herein, and refer to an animal that is the object of treatment, observation and/or experiment. “Animal” includes vertebrates and invertebrates, such as fish, shellfish, reptiles, birds, and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, the terms “ameliorating,” “teating,” “treatment,” “therapeutic,” or “therapy” do not necessarily mean total cure or abolition of the disease or condition. Any alleviation of any undesired signs or symptoms of a disease or condition, to any extent, can be considered amelioration, treatment and/or therapy. Furthermore, treatment may include acts that may worsen the patient's overall feeling of well-being or appearance.

The term “therapeutically effective amount/dose” or “inhibitory amount” is used to indicate an amount of an active compound, or pharmaceutical agent, that elicits the biological or medicinal response indicated. This response may occur in a tissue, system, animal or human and includes alleviation of the symptoms of the disease being treated.

The term “nucleic acids”, as used herein, may be DNA or RNA. Nucleic acids may also include modified nucleotides that permit correct read through by a polymerase and do not alter expression of a polypeptide encoded by that nucleic acid. The terms “nucleic acid” and “oligonucleotide” are used interchangeably to refer to a molecule comprising multiple nucleotides. As used herein, the terms refer to oligoribonucleotides as well as oligodeoxyribonucleotides. The terms shall also include polynucleosides (i.e., a polynucleotide minus the phosphate) and any other organic base containing polymer. Nucleic acids include vectors, e.g., plasmids, as well as oligonucleotides. Nucleic acid molecules can be obtained from existing nucleic acid sources, but are preferably synthetic (e.g., produced by oligonucleotide synthesis).

The phrase “nucleotide sequence” includes both the sense and antisense strands as either individual single strands or in the duplex.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It being further understood that the sequence includes the degenerate codons of the native sequence or sequences which may be introduced to provide codon preference in a specific host cell.

By “DNA” is meant a polymeric form of deoxyribonucleotides (adenine, guanine, thymine, or cytosine) in double-stranded or single-stranded form, either relaxed and supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence homologous to the mRNA). The term captures molecules that include the four bases adenine, guanine, thymine, or cytosine, as well as molecules that include base analogues which are known in the art.

A “gene” or “coding sequence” or a sequence which “encodes” a particular protein, is a nucleic acid molecule which is transcribed (in the case of DNA) and translated (in the case of mRNA) into a polypeptide in vitro or in vivo when placed under the control of appropriate regulatory or control sequences. The boundaries of the gene are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present so long as the selected coding sequence is capable of being replicated, transcribed and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence.

The term “operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

For the purpose of describing the relative position of nucleotide sequences in a particular nucleic acid molecule throughout the instant application, such as when a particular nucleotide sequence is described as being situated “upstream,” “downstream,” “5′,” or “3′” relative to another sequence, it is to be understood that it is the position of the sequences in the non-transcribed strand of a DNA molecule that is being referred to as is conventional in the art.

The term “homology” refers to the percent of identity between two polynucleotide or two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single-stranded-specific nuclease(s), and size determination of the digested fragments. Two DNA, or two polypeptide sequences are “substantially homologous” to each other when at least about 80%, preferably at least about 90%, and most preferably at least about 95% of the nucleotides or amino acids match over a defined length of the molecules, as determined using the methods above.

By “isolated” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

The terms “vector”, “cloning vector”, “expression vector”, and “helper vector” mean the vehicle by which a DNA or RNA sequence (e.g., a foreign gene) can be introduced into a host cell, so as to promote expression (e.g., transcription and/or translation) of the introduced sequence. Vectors include plasmids, phages, viruses, pseudoviruses, etc.

“Gene transfer” or “gene delivery” refers to methods or systems for reliably inserting foreign DNA into host cells.

As used herein, the term “transfection” is understood to include any means, such as, but not limited to, adsorption, microinjection, electroporation, lipofection and the like for introducing an exogenous nucleic acid molecule into a host cell. The term “transfected” or “transformed”, when used to describe a cell, means a cell containing an exogenously introduced nucleic acid molecule and/or a cell whose genetic composition has been altered by the introduction of an exogenous nucleic acid molecule.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry. Solid phase polypeptide synthesis procedures are well known in the art and further described by John Morrow Stewart and Janis Dillaha Young, Solid Phase Peptide Syntheses (2nd Ed., Pierce Chemical Company, 1984). Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing. In cases where large amounts of a polypeptide are desired, it can be generated using recombinant techniques such as described by Bitter et alt., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

As used herein, the term “tumor” refers to a malignant tissue comprising transformed cells that grow uncontrollably. A tumor may be benign (benign tumor) or malignant (malignant tumor or cancer). Tumors include leukemias, lymphomas, myelomas, plasmacytomas, and the like; and solid tumors. Examples of solid tumors include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, epidermoid carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, neuroglioma, and retinoblastoma.

As used herein the phrase “prostate cancer” refers to cancers of the prostate tissue and/or other tissues of the male genitalia, or reproductive or urinary tracts.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, “carrier” includes any and all solvents, dispersion media, vehicles, coatings, diluents, antibacterial and antifingal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

As used herein, the term “heterologous sequence or gene” means a nucleic acid (RNA or DNA) sequence, which is not naturally found in association with the nucleic acid sequences of the specified molecule.

### Antibodies

The detection of the expression profile of Claudin-1 in prostate cells may be carried out by any of several means well known to those of skill in the art. Some embodiments disclosed herein relate to methods of detecting Claudin-1 that is immunological in nature. “Immunological” refers to the use of antibodies (e.g., polyclonal or monoclonal antibodies) specific for Claudin-1. “Specific for Claudin-1” refers to antibodies that recognize Claudin-1 while not substantially cross-reacting with control samples containing other proteins. Antibodies specific for Claudin-1 include, but is not limited to, commercially available antibodies (e.g., antibodies available from Aviva System Biology, Santa Cruz Biotech, Inc., USBIO, LifeSpan, Novus Bio., Biotrend, Abnova, Cell Signaling, abcam, GenWay, antibodies-online, Zymed, and GeneTex) and those antibodies that can be produced by methods disclosed herein and by methods known in the art.

The term “antibody” includes immunoglobulin molecules and immunologically active determinants of immunoglobulin molecules, i.e., molecules that contain an antigen binding site which specifically binds (e.g., immunoreacts with) an antigen. Structurally, the simplest naturally occurring antibody (e.g., IgG) comprises four polypeptide chains, two copies of a heavy (H) chain and two of a light (L) chain, all covalently linked by disulfide bonds. Specificity of binding in the large and diverse set of antibodies is found in the variable (V) determinant of the H and L chains; regions of the molecules that are primarily structural are constant (C) in this set. Antibody includes polyclonal antibodies, monoclonal antibodies, whole immunoglobulins, and antigen binding fragments of the immunoglobulin.

The binding sites of the proteins that comprise an antibody, i.e., the antigen-binding functions of the antibody, are localized by analysis of fragments of a naturally-occurring antibody. Thus, antigen-binding fragments are also intended to be designated by the term “antibody.” Examples of binding fragments encompassed within the term antibody include: a Fab fragment consisting of the VL, VH, CL and CH1 domains; an F_{c} fragment consisting of the VH and CH1 domains; an Fᵥ fragment consisting of the V_{L} and V_{H} domains of a single arm of an antibody; a dAb fragment (Ward et al., 1989 Nature 341:544-546) consisting of a VH domain; an isolated complementarity determining region (CDR); and an F(ab′)₂ fragment, a bivalent fragment comprising two Fab′ fragments linked by a disulfide bridge at the hinge region. These antibody fragments are obtained using conventional techniques well-known to those with skill in the art, and the fragments are screened for utility in the same manner as are intact antibodies. The term “antibody” is further intended to include bispecific and chimeric molecules having at least one antigen binding determinant derived from an antibody molecule, as well as single chain (scFv) antibodies.

The term “single-chain Fv,” also abbreviated as “sFv” or “scFv,” refers to antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

In the diagnostic and prognostic assays of embodiments disclosed herein, the antibody can be a polyclonal antibody or a monoclonal antibody.

### Labels

In some contexts, the term “marker” refers to a nucleic acid fragment, a peptide, or a polypeptide (e.g., Claudin-1 and/or AMACR), which is differentially present in a sample taken from subjects (patients) having prostate cancer as compared to a comparable sample taken from subjects who do not have prostate cancer. In general, in order to be considered significantly under expressed, Claudin-1 may be detected as present in an amount of about 25 to about 75% or more below the known, standardized level of normal control tissue, or more preferably from about 50 to about 100% or more lower than the known, standardized level of normal control tissue. A “known, standardized level of normal control tissue” refers to that level detected in equivalent tissue derived from disease-free individuals. Further, such comparisons are typically made in comparison to a known negative control, such as tissue known to be devoid of the antigen being detected.

The phrase “differentially present” refers to differences in the quantity of a marker present in a sample taken from patients having prostate cancer as compared to a comparable sample taken from patients who do not have prostate cancer. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed. “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease can be affected by determining a level of a polynucleotide or a polypeptide in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

As used herein, the term “level” refers to expression levels of RNA and/or protein or to DNA copy number of a marker.

Typically the level of the marker in a biological sample obtained from the subject is different (i.e., increased or decreased) from the level of the same variant in a similar sample obtained from a healthy individual (examples of biological samples are described herein).

As used herein, “predetermined level” refers to the level of expression of a prostate cancer marker in normal, non-cancerous prostate tissue. In some embodiments, prostate cancer can be diagnosed by assessing whether Claudin-1 is expressed at a level that is less than or equal to 50%, 45%, 40%, 35%, 30%, 25%, 20%, 15%, 10%, 9%, 8%, 7%, 6%, 5%, 4%, 3%, 2%, 1% or 0% of a predetermined level. In other embodiments, prostate cancer can further be diagnosed by assessing whether AMACR expression varies from a predetermined level by greater than or equal to 50%, 55%, 60%, 65%, 70%, 75%, 80%, 85%, 90%, 91%, 92%, 93%, 94%, 95%, 96%, 97%, 98%, 99% or 100%.

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), lavage, and any known method in the art. Regardless of the procedure employed, once a biopsy/sample is obtained the level of the variant can be determined and a diagnosis can thus be made. For example, tissue sample may be obtained by biopsy or prostatectomy. The sample of cells or tissue can then be prepared and exposed to an antibody or a mixture of antibodies according to means which are known to those of skill in the art. Samples can then be paraffin embedded and sectioned for immunohistochemical analysis of gene expression.

Determining the level of the same variant in normal tissues of the same origin is preferably effected along-side to detect an elevated expression and/or amplification and/or a decreased expression, of the variant as opposed to the normal tissues.

A “test amount” of a marker refers to an amount of a marker in a subject's sample that is consistent with a diagnosis of prostate cancer. A test amount can be either in absolute amount (e.g., microgram/ml) or a relative amount (e.g., relative optical density [OD] or intensity of signals in pixels/mm²).

A “control amount” of a marker can be any amount or a range of amounts to be compared against a test amount of a marker. For example, a control amount of a marker can be the amount of a marker in a patient with prostate cancer or a person without prostate cancer. A control amount can be either in absolute amount (e.g., microgram/ml) or a relative amount (e.g., relative optical density [OD] or intensity of signals in pixels/mm²).

The term “detect” refers to identifying the presence, absence or amount of the object to be detected.

“label” includes any moiety or item detectable by spectroscopic, photo chemical, biochemical, immunochemical, or chemical means. For example, useful labels include fluorescent dyes, radionuclides, phosphors, electron-dense reagents, enzymes, enzyme products (e.g., chromagens catalytically processed by horseradish peroxidase or alkaline phosphatase commonly used in an ELISA or immunocytochemistry), biotin-avidine and -streptavadin/polymer systems, dioxigenin, colloidal dye substances, fluorochromes, reducing substances, latexes, metals, particulates, dansyl lysine, antibodies, protein A, protein G, chromophores, haptens, and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by avidine/streptavadin. The label may be directly or indirectly detectable. Indirect detection can involve the binding of a second label to the first label, directly or indirectly. For example, the label can be the ligand of a binding partner, such as biotin, which is a binding partner for avidine/streptavadin, or a nucleotide sequence, which is the binding partner for a complementary sequence, to which it can specifically hybridize. The binding partner may itself be directly detectable, for example, an antibody may be itself labeled with fluorescent molecules and/or enzymes (e.g., HRP or alkaline phosphatase). The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, flow cytometry and/or microscopical analysis with computer-algorithm assisted software(s).

Exemplary detectable labels, optionally and preferably for use with immunoassays, include but are not limited to magnetic beads, fluorescent dyes, radiolabels, enzymes, chromagens catalytically processed by enzymes (e.g., horseradish peroxide (HRP), alkaline phosphatase and others commonly used in an ELISA and immunocytochemisry), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Visualization of enzymes, (e.g., HRP or alkaline phosphatase), can be achieved by means of using the enzymatic activity of the enzyme, e.g., the oxidative-catalytic enzymatic activity of HRP or Alkaline phosphatase, to process and precipitate a substrate-chromogen. The final reaction product may be soluble in buffer or ethanol and may require stabilization to prevent fading. Chromogens that can be used include, but are not limited to 3,3′-diaminobenzidine tetrahydrochloride (DAB), Betazoid DAB, Cardassian DAB, 3,3′,5,5′-tetramethylbenzidine (TMB), benzidine dihydrochloride (BDHC) and p-phenylenediamine dihydrochloride with pyrocatechol (PPD-PC), 4-chloro-1-naphthol (4C1N), 3-amino-9-ethylcarbazole (AEC) and o-phenylenediamine (OPD), DAB-NI (Vector Laboratories), VECTOR® VIP (Vector Laboratories), VECTOR® SG (Vector Laboratories), VECTOR® RED (Vector Laboratories), VECTOR® BLACK (Vector Laboratories), VECTOR® BLUE (Vector Laboratories), BCIP/NBT (Vector Laboratories), Glucose oxidase NBT (Vector Laboratories), Glucose oxidase TNBT (Vector Laboratories), and Glucose oxidase INT (Vector Laboratories), Bajoran Purple, Romulin AEC, Ferangi Blue and Vulcan Fast Red (Biocare Medical Inc.). Some chromogens (e.g., Bajoran Purple and VECTOR® RED) may also be used in double and triple stain procedures, nitrocellulose blots, and can be viewed by both bright- and darkfield microscopy. The visualization of the reaction product can be further improved by intensification with metal salts. At the light microscopic level, this intensification can enable color differentiation between distinct markers (see, e.g., van der Want et al., Tract-tracing in the nervous system of vertebrates using horseradish peroxidase and its conjugates: tracers, chromogens and stabilization for light and electron microscopy. Brain Res Brain Res Protoc. 1997 August; 1(3):269-79, which is hereby incorporated by reference in its entirety). In addition, the amounts of these precipitates can be semi-automatically or automatically quantified by algorithm based software (e.g., Aperio Technology Inc, Vista, Calif.). Visualization can be achieved by using combinations of detectable labels in embodiments disclosed herein. For example, HRP can be used with alkaline phosphatase and visualized by microscopy (e.g., bright—or dark-field microscopy) to differentiate between two or more distinct markers.

Examples of fluorescent dyes include, but are not limited to, 7-Amino-actinomycin D, Acridine orange, Acridine yellow, Alexa Fluor dyes (Molecular Probes), Auramine O, Auramine-rhodamine stain, Benzanthrone, 9,10-Bis(phenylethynyl)anthracene, 5,12-Bis(phenylethynyl)naphthacene, CFDA-SE, CF SE, Calcein, Carboxyfluorescein, 1-Chloro-9,10-bis(phenylethynyl)anthracene, 2-Chloro-9,10-bis(phenylethynyl)anthracene, Coumarin, Cyanine, DAPI, Dark quencher, Dioc6, DyLight Fluor dyes (Thermo Fisher Scientific), Ethidium bromide, Fluorescein, Fura-2, Fura-2-acetoxymethyl ester, Green fluorescent protein and derivatives, Hilyte Fluor dyes (AnaSpec), Hoechst stain, Indian yellow, Luciferin, Perylene, Phycobilin, Phycoerythrin, Phycoerrobilin, Propidium iodide, Pyranine, Rhodamine, RiboGreen, Rubrene, Ruthenium(II) tris(bathophenanthroline disulfonate), SYBR Green, Stilbene, Sulforhodamine 101, TSQ, Texas Red, Umbelliferone, and Yellow fluorescent protein.

Examples of phsosphors include, but are not limited to Phosphor, Anthracene, Barium fluoride, Bismuth germanate, Cadmium sulfide, Cadmium tungstate, Gadolinium oxysulfide, Lanthanum bromide, Polyvinyl toluene, Scheelite, Sodium iodide, Stilbene, Strontium aluminate, Yttrium aluminium garnet, Zinc selenide, Zinc sulfide

Examples of radionuclides include, but are not limited to, ³²P, ³³P, ⁴³K, ⁴⁷Sc, ⁵²F⁵²Co, ⁶⁴Cu, ⁶⁷Ga, ⁶⁷Cu, ⁶⁸Ga, ⁷¹Ge, ⁷⁵Br, ⁷⁶Br, ⁷⁷Br ⁷⁷As, ⁷⁷Br, 7R/⁸¹MKr, ⁸⁷MSr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Antibodies can be radiolabeled, for example, by the Iodogen method according to established methods.

A label may be chemically coupled directly to an antibody (i.e., without a linking group) through an amino group, a sulfhydryl group, a hydroxyl group, or a carboxyl group.

In some embodiments, a label can be attached to an antibody via a linking group. The linking group can be any biocompatible linking group, where “biocompatible” indicates that the compound or group can be non-toxic and may be utilized in vitro or in vivo without causing injury, sickness, disease, or death. The label can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl butanoate (SBA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide (or other epitope), refers to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

### Immunoassays

In other embodiments, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample. Immunoassays can be conducted in vitro or in vivo.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, an immunohistochemical assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture. An antibody can be labeled either before, during or after the incubation step.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

In some embodiments, antibodies can be used which specifically interact with the polypeptides of the embodiments disclosed herein and not with wild type proteins or other isoforms thereof, for example. Such antibodies can be directed, for example, to the unique sequence portions of the polypeptide variants, including but not limited to bridges, heads, tails and insertions described in greater detail below.

Radio-immunoassay (RJA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Immunohistochemical analysis: This method involves detection of a substrate in situ in fixed cells or tissues by substrate specific antibodies. The substrate specific antibodies may be enzyme linked or linked to fluorophores. Detection can be by microscopy and subjective evaluation. Detection can be automated. If enzyme linked antibodies are employed, a calorimetric reaction may be required.

Immobilization may be required and may be accomplished by immobilizing the protein or peptide to a solid surface, such as a microtiter well, or by binding the protein to immobilized antibodies. In an embodiment, the Claudin-1 is bound to an immobilized first antibody. A second labeled antibody, also specific for the Claudin-1, or specific for the first antibody, is then bound, unbound material is washed away, and the complex is detectable due to the immobilized label of the second antibody. Such assays are well-known to those of skill in the art and include such assays as simultaneous sandwich, forward sandwich and reverse sandwich immunoassays, terms which are well-known to those of skill in the art. Many solid phase immunoabsorbents for immobilization are known and can be used. Well-known immunoabsorbents include beads formed from glass, polystyrene, polypropylene, dextran, nylon and other material; and tubes formed from or coated with such materials, and the like. The immobilized antibodies may be covalently or physically linked to the solid phase immunosorbent by techniques such as covalent bonding via an amide or ester linkage or by absorption. In each of the above assays, the exact details of the assay protocol, such as time and temperature of incubation, may vary according to the concentration of antibodies used, the source and form of the sample, the affinity of the antibodies for their target molecules, etc. In an embodiment, an ELISA assay may be carried out as follows: 96-well microtiter plates are coated with a monoclonal first antibody specific for Claudin-1. The first antibody is immobilized in the wells. Standards and samples are pipetted into wells in, for example, duplicate or triplicate, and the Claudin-1 present in the standards and samples will be bound by the immobilized antibody. The standards are composed of known concentrations of Claudin-1, which is known to be cross-reactive with the first antibody. After incubation at room temperature for 2 hours, the wells are washed with an appropriate buffer to remove any unbound substances. Then a second enzyme-linked polyclonal (or monoclonal) antibody specific for Claudin-1 (or for the primary antibody) is added to the wells. After a 1 hour incubation at room temperature, the wells are again washed with an appropriate buffer to remove unbound antibody-enzyme reagent, and a solution which contains a substrate for the enzyme is added to the wells. The substrate is such that when it is acted on by the enzyme, a characteristic color is produced. Color will develop in proportion to the amount of enzyme present in the wells, which is directly proportional to the amount of: bound claudin. After an appropriate period of time, the color development is stopped and the intensity of the color will be measured spectrophotometrically. The amount of claudin in the samples can be determined by comparing the color intensity of the sample wells to that of the control wells which contain a known amount of Claudin-1.

Other means of detecting the expression profile of Claudin-1 include but are not limited to, for example, detection of mRNA encoding the protein. Those of skill in the art are well acquainted with methods of mRNA detection, e.g., via the use of complementary hybridizing primers (e.g., labeled with radioactivity or fluorescent dyes) with or without polymerase chain reaction (PCR) amplification of the detected products, followed by visualization of the detected mRNA via, for example, electrophoresis (e.g., gel or capillary); by mass spectroscopy; etc. Any means of detecting the presence of the mRNA in an amount lower than normal or baseline control (or to detect the absence of the mRNA For example, an immunoassay can measure the level of gene expression (e.g., Claudin-1) or activity by measuring the level of mRNA. The level of mRNA may also be measured, for example, using ethidium bromide staining of a standard RNA gel, Northern blotting, primer extension, or nuclease protection assay.

Other immune assays which may be utilized include, but are not limited to agglutination methods, precipitation methods, immunodiffusion methods, immunoelectrophoresis methods, nephelometry, gel electrophoresis followed by Western blot, dot blots, affinity chromatography, immune-fluorescence, and the like. In addition, other methods of detection of peptides (e.g., Claudin-1) known to those of skill in the art may be used, such as gas chromatography/mass spectrometry, HPLC, and gel electrophoresis followed by sequencing.

### Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference in its entirety.

### Monitoring Cancer Therapy

The phrase “monitoring cancer therapy” refers to determining the relative amount of cancer cells in the body of a patient before, during and/or after anti-cancer therapy.

Some embodiments disclosed herein relate to methods for monitoring the progress or efficacy of cancer therapy in a subject. Subjects identified as having cancer and undergoing cancer therapy can be administered labeled Claudin-1 antibodies.

Subjects can be administered a labeled antibody before the onset of treatment or during treatment. Cells containing the label can be assayed for and this measurement can be compared to one obtained at a subsequent time during the therapy and/or after therapy has been completed. In this way, it is possible to evaluate the inhibition of cancer cell proliferation, and the effectiveness of the therapy. Since only living cancer cells will be detected via the labeled antibody, the therapy can continue until a minimal amount of label is detected.

Some embodiments disclosed herein also relate to methods for determining the amount of cancer cells present in a subject. By detecting the label, one can determine whether cancer cells are present within the subject and the amount of label measured is proportional to the amount of cancer cells present in the subject.

### Kits

Some embodiments disclosed herein provide for a kit for detecting a cell-proliferative disorder (e.g. prostate cancer) comprising an agent which binds specifically to a Claudin-1 marker and instructions for use.

In one embodiment, the kit may comprise a reference sample, e.g., a negative and/or positive control. In that embodiment, the negative control would be indicative of a normal cell type and the positive control would be indicative of cancer. Such a kit may also be used for identifying potential candidate therapeutic agents for treating cancer. In one embodiment, the first binding moiety is labeled. In one embodiment, the kit further comprises a second binding moiety which binds specifically to the first binding moiety.

The above mentioned kit can be used for the detection of any cell-proliferative cancer including, without limitation, breast cancer, ovarian cervical cancer, prostate cancer, colon cancer, lung cancer, skin cancer, leukemia, lymphoma, melanoma or any other type of cancer. In one embodiment the kit is for the detection of prostate cancer.

The kit may also be used to determine the aggressiveness or grade of cancer, e.g., prostate cancer.

In one embodiment, the binding moiety in the kit can be an antibody or fragment thereof which specifically binds to Claudin-1. Antibodies and binding fragments thereof can be lyophilized or in solution. Additionally, the preparations can contain stabilizers to increase the shelf-life of the kits, e.g., bovine serum albumin (BSA). Wherein the antibodies and antigen binding fragments thereof are lyophilized, the kit can contain further preparations of solutions to reconstitute the preparations. Acceptable solutions are well known in the art, e.g., PBS. In one embodiment, the antibody is a polyclonal antibody, a monoclonal antibody, a humanized antibody, a chimeric antibody, a recombinant antibody, or fragment thereof.

In some embodiments, the kits can further include the components for an immunohistochemical assay for measuring Claudin-1 and fragments thereof. In some embodiments, the kits can further include the components for an ELISA assay for measuring Claudin-1 and fragments thereof. Samples to be tested in this application include, for example, blood, serum, plasma, urine, lymph, tissue and products thereof.

Alternatively, the kits can be used in immunoassays, such as immunohistochemistry to test subject tissue biopsy sections. The kits may also be used to detect the presence of a Claudin-1 marker in a biological sample obtained from a subject using immunohistocytochemistry.

The compositions of the kit of the present invention can be formulated in single or multiple units for either a single test or multiple tests.

Embodiments disclosed herein further provide for a kit for use in, for example, the screening, diagnosis or monitoring of prostate cancer. Such a kit may comprise an antibody to Claudin-1, a reaction container, various buffers, secondary antibodies, directions for use, and the like. In these kits, antibodies may be provided with means for binding to detectable marker moieties or substrate surfaces. Alternatively, the kits may include antibodies already bound to marker moieties or substrates. The kits may further include positive and/or negative control reagents as well as other reagents for carrying out diagnostic techniques. For example, kits containing antibody bound to multiwell microtiter plates can be provided. The kit may include a standard or multiple standard solutions containing a known concentrations of Claudin-1 or other proteins for calibration of the assays. A large number of control samples is assayed to establish the threshold, mode and width of the distribution of Claudin-1 in normal cells and tissues against which test samples are compared. These data is provided to users of the kit. In some embodiments, the kit can also include a reagent (e.g., an antibody) that detects the presence of alpha-methylacyl coenzyme A racemase (AMACR).

### EXAMPLES

### Example 1

### Immunohistochemical Analysis of Claudin-1 Expression in Normal, Premalignant and Malignant Human Prostatic Tissues

To characterize the expression of Claudin-1 in human prostate, tissue microarrays (TMAs) were employed which included trans-urethral retrograde prostatectomy (TURP) and radical prostatectomy specimens from 66 patients with locally confined (stage T2) and locally advanced disease (T3). Tissue samples comprised elements of benign prostatic hyperplasia (BPH; n=38), prostatic intraepithelial neoplasia (PIN; n=11), and prostate adenocarcinoma (n=41). Prostate cancer TMAs were constructed, as described previously (Krajewska M. et al. 2005 Clin. Cancer Res. 11:5462-5471), using blocks of annotated prostate cancer patients from the Sidney Kimmel Center. An Institutional review Board (IRB)—approved protocol was used to retrieve archival paraffin blocks from the Sharp HealthCare hospitals of San Diego.

Gleason score data were available for all tumors, while clinical stage information (T2-T3) (according to International Union against Cancer criteria) was known for 48% of patients. In addition, non-neoplastic prostate epithelium from 9% of cases was available for comparison of protein expression in non-transformed versus neoplastic epithelium.

Dewaxed tissue sections were immunostained using an automated immunostainer (DAKO Universal Staining System) and employing the Envision-Plus-horseradish peroxidase system (DAKO) (Krajewski S. et al. 1999 PNAS USA 96:5752-5757). Rabbit polyclonal anti-Claudin-1 antibody (Zymed) was used at 1.25 μg/mL. Also employed for immunostaining were mouse monoclonal antibodies to Bcl-2, p63, and keratin 34βE12 (high molecular weight keratin) and rabbit monoclonal antibody to AMACR (DakoCytomation).

For double-labeling procedure, tissue sections were stained as above using Claudin-1 rabbit polyclonal antiserum (SG chromagen, Vector Lab. Inc; black) followed by mouse monoclonal antibodies to Bcl-2, p63, or keratin 34βE12 (DAB chromagen, DakoCytomation), using Nuclear Red (DakoCytomation) for counterstaining.

An automated image analysis system (Aperio Technology Inc., Vista Calif.) was employed to visualize Claudin-1 and Bcl-2, p63, 34βE12 or AMACR stainings separately, applying a color deconvolution algorithm (Ruifrok A. C. et al. 2001 Anal. Quant. Cytol. Histol. 23:291-299).

Quantification of immunohistochemical staining was performed using color translation and an automated thresholding algorithm (Aperio Technology Inc, Vista Calif.).

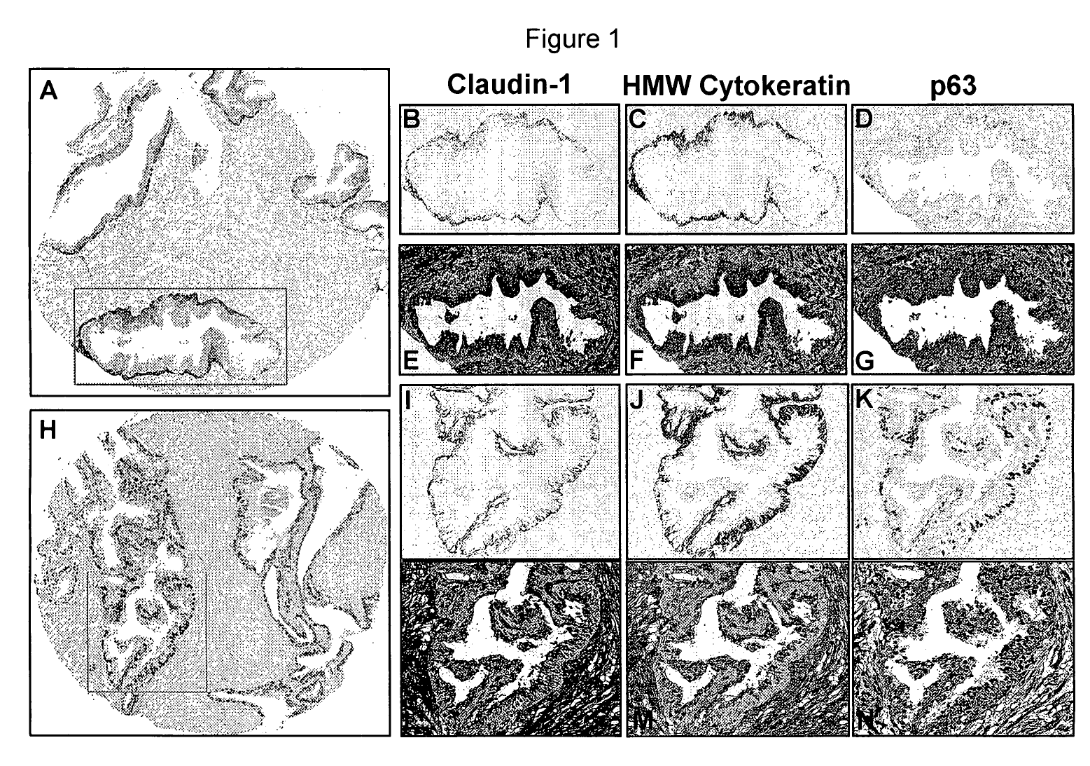

In normal prostatic epithelium, the Claudin-1 immunostaining was detected exclusively in the basal cell layer, showing cytosolic and membranous intracellular localization. No staining was observed in the secretory epithelial cells or in stroma. A comparison of Claudin-1 immunostaining with 34βE12, p63, and Bel-2 immunolabeling of serial sections, as well as double-labeling with monoclonal antibodies to HMW, p63 (FIG. 1) or Bcl-2, confirmed that the Claudin-1 immunopositive cells were in fact basal cells. TMA slides containing prostate specimens were double stained with the Claudin-1 (SG, black) and HMW cytokeratin antibodies (DAB) (FIG. 1, panels A, H) or with the Claudin-1 (SG) and p63 antibodies (DAB) and counterstained with Nuclear Red. The black (FIG. 1, panels B, I) and brown (FIG. 1, panels C, J for HMW cytokeratin; FIG. 1, panels D, K for p63) colors were separated in the annotated regions using a color deconvolution algorithm (Aperio). Quantification of immunohistochemical staining for Claudin-1 (FIG. 1, panels E, L), HMW cytokeratin (FIG. 1, panels F, M) and p63 (FIG. 1, panels G, N) was performed using color translation and an automated thresholding algorithm (Aperio). Claudin-1 colocalized with HMW cytokeratin or p63 in basal cells. 10× and 15× digital zooms were applied for the presented images (FIG. 1, panels A, H, and FIG. 1, panels B-D, E-G, I-N, respectively). In addition, Claudin-1 protein was detected in the perineum of peripheral nerves, consistent with prior reports (Pummi, K. P. et al. 2004 J. Histochem. Cytochem. 52:1037-1046).

BPH specimens retained a normal Claudin-1 staining pattern, whereas increasing grades of prostatic intraepithelial neoplasia (PIN) demonstrated intermittent Claudin-1 labeling, reflecting progressive disruption of the basal cell layer. Similarly, discontinuous Claudin-1 staining was observed in some normal-appearing glands surrounded by inflammatory cells.

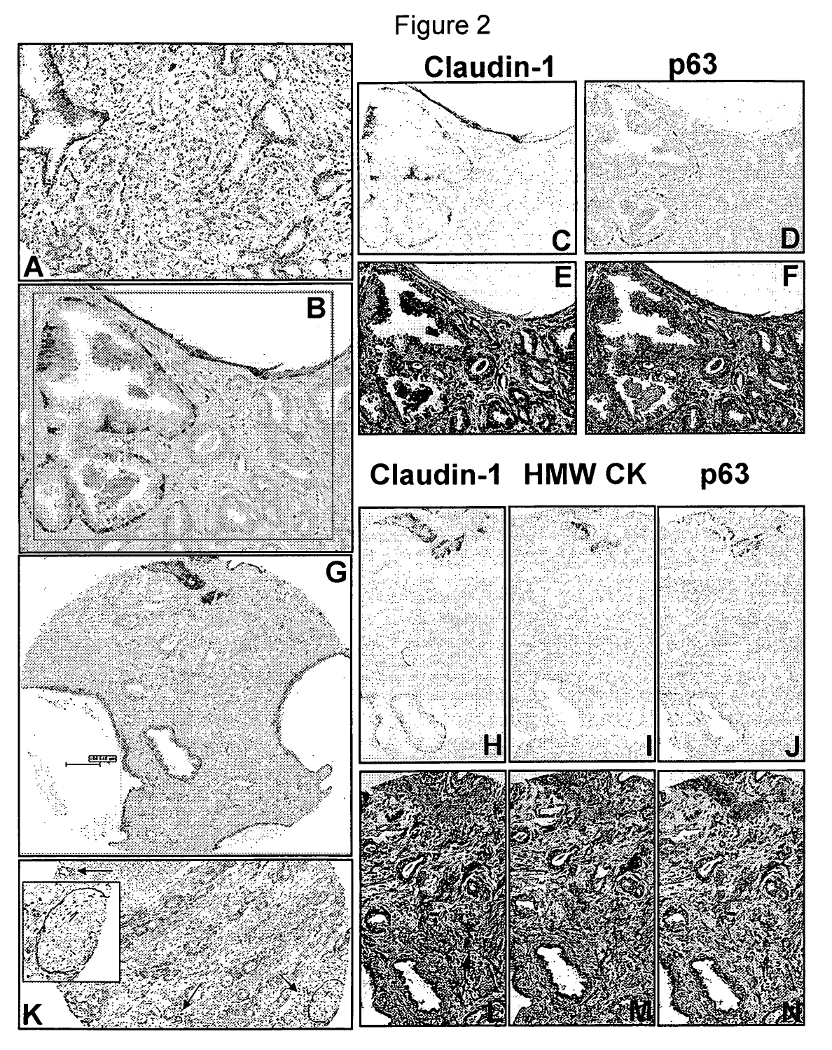

Using single—(FIG. 2, panel A—Claudin-1), or double-labeling (FIG. 2, panels B, G—Claudin-1 and HMW cytokeratin), bright-field and chromagen based indirect immunocytochemistry visualized the presence of Claudin-1 protein in benign glands but not in the adenocarcinoma. Either in single or double marker ICH using variations of secondary antibodies and chromagens the specific discrimination of two or more proteins was achieved and visualized either in brown (DAB (DakoCytomation Inc.)) or in black-gray colors (SG (Vector Lab. Inc.)). In addition, using Scanscope virtual pathology system (Aperio Technologies, Inc) equipped in a color deconvolution algorithm we could confirm microscopical observation performing color separation and quantitation of immune products in the annotated regions (SG (Vector Labs, Inc.)—black, FIG. 2, panels C, H; DAB (DakoCytomation, Inc.)—brown, FIG. 2, panels D, I, J). Quantification of immunohistochemical staining for claudin-1 (FIG. 2, panels E, L), HMW cytokeratin (FIG. 2, panel M) and p63 (FIG. 2, panels F, N) was performed using color translation and an automated thresholding algorithm (Aperio). Claudin-1 protein was immunodetected in the perineurium of peripheral nerves, which is a known internal control of claudin-1 immunostaining (FIG. 2, panel K; inset). For the better presentation of the specificity for novel versus known biomarkers, all areas presented on the panels in FIG. 2 contained tumor with a mixture of few preserved normal prostatic glands.

In 98% (47/48) of prostate cancer cases, malignant cells were uniformly Claudin-1 immunonegative (e.g., as shown in FIG. 2). Among these 47 Claudin-1 negative tumors, 8 (17%) showed positive staining with Bcl-2 antibody, excluding poor tissue processing as an explanation for the immunonegativity. The one low grade adenocarcinoma displaying any immunopositivity for Claudin-1 exhibited only weak intensity staining in occasional cells (approximately 2%). Double labeling with alpha-methylacyl coenzyme A racemase (AMACR), an enzyme selectively expressed in neoplastic glandular epithelium, demonstrated lack of Claudin-1 and AMACR co-expression. Thus, the immunostaining of these proteins were mutually exclusive. Therefore, it was shown that Claudin-1 expression is uniformly lost in prostate cancers.

The experiments above demonstrated that Claudin-1 expression in human prostate is confined to the basal cell layer, and that essentially all primary prostate adenocarcinomas lack expression of this TJ protein. Loss of claudin-1 protein expression in prostate cancer is consistent with the notion that disruption of the tight junctions is associated with loss of cohesion, lack of differentiation, and invasiveness during tumorigenesis (reviewed in Morin P. J. 2005 Cancer Res. 65:9603-9606).

Epithelial-mesenchymal transition (EMT) is a recently recognized phenomenon associated with epithelial carcinogenesis (reviewed in Yang, J. et al. 2006 Cancer Res. 66:4549-4552). The EMT-inducing transcription factors Snail and Slug were recently implicated as potential repressors of Claudin-1 expression ((Martinez-Estrada O. M. et al. 2006 Biochem J. 1394:449-457). In addition, Claudin-1 was recently identified as a possible target of β-catenin/Tcf signaling in colorectal carcinogenesis (Miwa N. et al. 2001 Oncol. Res. 12:469-476; Dhawan P. et al. 2005 J. Clin. Invest. 115:1765-1776). Cooperation between Snail and LEF-1 transcription factors has been shown to be essential for TGF-β1-induced EMT associated with loss of Claudin-1 and E-cadherin (Medici D. et al. 2006 Mol. Biol. Cell. 17:1871-1879). Absence of Claudin-1 protein expression provides a marker for aiding in pathological diagnosis of prostate cancers.

### Example 2

### Determination of Prostate Cancer using Claudin-1 and AMACR

Using immunohistochemistry, the expression of claudin-1 was investigated in prostate tissue samples arranged in a tissue microarray (TMA) format and comprising elements of normal prostatic epithelium (n=6), benign prostatic hyperplasia (BPH; n=38), prostatic intraepithelial neoplasia (PIN; n=11), and prostate adenocarcinoma (n=48). The claudin-1 expression pattern was compared with that of the basal cell-specific markers, p63, and HMW cytokeratin (34βE12) as well as prostate carcinoma marker a-methylacyl-CoA racemase (AMACR), by employing double-labeling technique, automated image analysis system, and color deconvolution, as well as color translation and automated thresholding algorithms.

In the benign prostatic epithelium, pronounced claudin-1 expression was observed in the basal cell layer with no staining in luminal cells. AMACR-positive prostate adenocarcinoma specimens from 96% (46/48) of patients did not contain claudin-1 immunostaining, whereas the remaining cases (4%) presented weak claudin-1 signal in single scattered neoplastic glands. Thus, the claudin-1 immunostaining was specific for normal basal cells, while the AMACR immunostaining was positive for cancer cells.

These results demonstrate that claudin-1 and AMACR double-immunohistochemistry can be applied as a new diagnostic tool for distinguishing malignant from benign epithelial lesions of the prostate. It can be utilized in a conventional, human eye-based pathological diagnosis or in semi-automated computer-assisted analysis using the scanning device and the morphometry software.

Other markers that can be used in combination dual staining with Claudin-1 in embodiments related to methods for the diagnosis and prognosis of prostate cancer include, but are not limited to, AR, PSA, PSMA (prostate specific membrane antigen), EPCA-2 (Early Prostate Cancer Antigen-2), PSAP (Prostate Specific Alkaline Phosphatase), EMA (epithelial membrane antigen), S-100 protein and MMP 1 (metalloproteinase 1).

### Example 3

### Determination of Prostate Cancer in a Patient

A patient at risk for prostate cancer is given a needle biopsy to determine whether or not he has prostate cancer. The biopsy sample is tested with a double immunohistochemistry analysis to determine if the cells from the biopsy are positive for claudin-1 or AMACR. The patient is found to have positive staining for claudin-1 and no staining for AMACR. Thus, the patient is determined to not have prostate cancer.

### Example 4

### Determination of Prostate Cancer in a Patient

A second patient at risk for prostate cancer is given a needle biopsy to determine whether or not he has prostate cancer. The biopsy sample is tested with a double immunohistochemistry analysis to determine if the cells from the biopsy are positive for claudin-1 or AMACR. The patient is found to have negative staining for claudin-1 and positive staining for AMACR. Thus, the patient is determined to have prostate cancer.

While the embodiments have been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A method for diagnosing prostate cancer in a patient, comprising:
providing a patient at risk for prostate cancer;
determining a level of Claudin-1 expression in a prostate sample from said patient; and
assessing whether Claudin-1 is expressed at a level which is lower than a predetermined level, thereby diagnosing prostate cancer in the patient.

2. The method of claim 1 wherein determining the level of Claudin-1 expression comprises contacting said prostate sample with an anti-Claudin-1 antibody.

3. The method of claim 1 further comprising obtaining said prostate sample from said patient.

4. The method of claim 3, wherein said prostate sample is a biopsy tissue sample.

5. The method of claim 1, further comprising grading said prostate sample wherein said grade is high if Claudin-1 expression is low.

6. A method for diagnosing prostate cancer in a patient, comprising:
providing a patient at risk for prostate cancer;
determining a level of Claudin-1 expression in a prostate sample from said patient;
determining a level of expression of alpha-methylacyl-CoA racemas (AMACR) in said prostate sample from said patient; and
assessing whether Claudin-1 is expressed at a level which is lower than a predetermined level, and whether AMACR is expressed at a level that is higher than a predetermined level, thereby diagnosing prostate cancer in the patient.

7. The method of claim 6, wherein determining the level of Claudin-1 expression comprises determining the level of Claudin-1 mRNA.

8. The method of claim 6, wherein determining the level of Claudin-1 expression comprises contacting said prostate sample with anti-Claudin-1 antibodies.

9. The method of claim 8, wherein said anti-Claudin-1 antibodies are polyclonal antibodies.

10. The method of claim 8, wherein said anti-Claudin-1 antibodies are monoclonal antibodies.

11. The method of claim 6, wherein determining a level of expression of AMACR comprises contacting said AMACR with anti-AMACR antibodies.

12. A method for monitoring the progress of a prostate cancer therapy in a subject comprising:
identifying a subject with prostate cancer;
providing said subject a prostate cancer therapy;
administering to said subject a detectable amount of a Claudin-1 antibody that comprises a detectable label; and
determining the presence or amount of Claudin-1 antibody bound to prostate cancer cells in said subject, before a treatment with said prostate cancer therapy and during or after a period of said treatment.

13. The method of claim 12, wherein determining the level of Claudin-1 expression comprises contacting said prostate sample with anti-Claudin-1 antibodies.

14. The method of claim 13, wherein said anti-Claudin-1 antibodies are monoclonal antibodies.

15. The method of claim 13, wherein said anti-Claudin-1 antibodies are polyclonal antibodies.

16. The method of claim 12, wherein determining a level of expression of AMACR comprises contacting said AMACR with anti-AMACR antibodies.

17. A kit comprising an agent that specifically detects Claudin-1 and instructions for using the kit components to determine the level of Claudin-1 in a person at risk for prostate cancer.

18. The kit of claim 17, wherein said agent that specifically detects Claudin-1 is an antibody that binds to Claudin-1.

19. The kit of claim 17, further comprising an agent that specifically detects alpha-methylacyl-CoA racemas (AMACR).
